(19) United States
(12) Patent Application Publication
(10) Pub. No.: US 2005/0038348 A1
(43) Pub. Date: Feb. 17, 2005

(54) Flashing jewelry heartbeat monitor with multiple lights

(76) Inventors: Kenneth Avicola, Livermore, CA (US); Richard G. Morton, San Diego, CA (US); John R. Ross, Del Mar, CA (US)

Correspondence Address: JOHN R. ROSS, PO Box 2138, DEL MAR, CA, 92014, US

(21) Appl. No.: 10/789,474

(22) Filed: Feb. 27, 2004

Related U.S. Application Data
(60) Provisional application No. 60/451,009, filed on Feb. 28, 2003.
(60) Provisional application No. 60/516,101, filed on Nov. 3, 2003.

(51) Int. Cl. A61B 5/02

(52) U.S. Cl. 600/502

(57) ABSTRACT

Jewelry, such as finger rings and earrings, which flash in synchronism with the wearer's heartbeat. A pulsed IR signal is directed into the wearer's tissue and a reflected or transmitted signal is monitored to determine when the wearer's heart beats at which time one or more light emitting sources in the jewelry flashes. The monitored signal is utilized to determine the wearer's heart rate. At least two light emitting sources are provided one of which flashes with each heart beat and the other flashes when the heart rate reaches or exceeds a predetermined range or increases faster than a predetermined rate. Preferred embodiments include three visible LED's (red, green and blue) and a micro-processor which calculates pulse rate and causes the red LED to blink on each pulse, the green LED to blink on each pulse when the wearer's pulse rate is greater a first threshold and the blue LED to blink on each pulse when the wearer's pulse rate is greater than a second threshold corresponding to extreme excitement. These threshold values may correspond to increased heart rates of typical persons engaged in exercise and love-making. The monitor may also be self calibrating to adjust the thresholds based on measurements of the wearer's heart rate over extended periods which would include periods of rest as well as periods of exertion or excitement. Other preferred embodiments vary the brightness of the LED's depending on the estimated blood pressure that also increases by about the same degree as pulse rate.

This application claims the benefit of Provisional Applications Ser. No. 60/516,101 filed Nov. 3, 2003 and Ser. No. 60/451,009 filed Feb. 28, 2003. The present invention relates to jewelry and heartbeat monitors.

### BACKGROUND OF THE INVENTION

U.S. Pat. No. 6,277,079 issued to the present inventors discloses a flashing earring heartbeat monitor. The '079 patent is incorporated herein by reference. In the background section of that patent is disclosed a well-known technique for monitoring heartbeat. Infrared light is transmitted into tissue and the reflected light is monitored to determine heart beat. This works because changing quantities of blood in the tissue with each heartbeat will affect the amount of light reflected from the tissue. In that patent Applicants described an earring (see FIGS. 1 and 2, which were FIGS. 2 and 3 in the '079 patent) which used that basic technique to make an earring which would flash with each heartbeat. Applicants summarized the invention as follows:

“The . . . invention provides an earring that flashes in synchronism with the wearer's heartbeat. A pulsed IR LED/photocell combination is built into an earring along with a comparator and a visible light-emitting source. The comparator determines when the heart has beat from the variation in the signal from the photocell and transmits a signal to a solid state switch to turn on the visible light-emitting source. Thus, the light emitting source flashes once for each heart beat. In a preferred use of the ... invention a lover is able to determine when his or her partner is excited by observing the rate at which the partner's earring flashes. The invention may also be used for medical monitoring of patients.”

Heart rates are known to vary substantially with age and activity. Resting heart rates are typically in the range of about 50 to 70 beats per minute. A rule of thumb is: Maximum Heart Rate=220−Age. The range of variation gets smaller with age. The maximum heart rate ranges from about 100 to 200 for 20 year old people and from about 75 to about 150 for 70 year old people. Heart rates increase toward the maximum rates during strenuous exercise and during periods of excitement including sexual excitement. FIGS. 4A and 4B are graphs extracted from page 596 of Sexual Behavior in the Human Female by the Staff of the Institute for Sex Research, Indiana University, Alfred C. Kinsey et al Research Associates, Published by W. B. Sanders, Philadelphia. The subjects were a married couple. The graphs show heart rate ranges for the female from about 90 during the period preceding love making to 140 during lovemaking down to about 75 during sleep. For the male the corresponding ranges were about 90, 140 and 72. The female experienced four periods of extreme excitement. The male experienced only one.

Applicants have been informed that the potential market for these flashing earring heartbeat monitors may be in the many millions and that other jewelry monitoring heartbeat my also be very popular. What is needed are an improved versions of the earring described in the '079 patent and other jewelry applications of improved versions of that invention.

### SUMMARY OF THE INVENTION

The present invention provides jewelry, such as finger rings and earrings, which flash in synchronism with the wearer's heartbeat. A pulsed IR signal is directed into the wearer's tissue and a reflected or transmitted signal is monitored to determine when the wearer's heart beats at which time one or more light emitting sources in the jewelry flashes. The monitored signal is utilized to determine the wearer's heart rate. At least two light emitting sources are provided one of which flashes with each heart beat and the other flashes when the heart rate reaches or exceeds a predetermined range or increases faster than a predetermined rate. Preferred embodiments utilize inexpensive off-the-shelf reflection sensors developed for card readers each of which includes both an IR emitter and a phototransistor IR detector. Preferred embodiments include a power-up-on-skin-contact feature to preserve battery power. Embodiments include a variety of color LED's and a variety of types of jewelry are proposed as heartbeat monitors. Disclosed are detailed instructions describing working prototype earrings built by Applicants and their licensees and descriptions for low cost fabrication of embodiments of the present invention using application specific integrated circuit (ASIC) technology and surface mount technology. Preferred embodiments include three visible LED's (red, green and blue) and a micro-processor which calculates pulse rate and causes the red LED to blink on each pulse, the green LED to blink on each pulse when the wearer's pulse rate is greater a first threshold and the blue LED to blink on each pulse when the wearer's pulse rate is greater than a second threshold corresponding to extreme excitement. These threshold values may correspond to increased heart rates of typical persons engaged in exercise and love making. The monitor may also be self calibrating to adjust the thresholds based on measurements of the wearer's heart rate over extended periods which would include periods of rest as well as periods of exertion or excitement. Other preferred embodiments vary the brightness of the LED's depending on the estimated blood pressure that also increases by about the same degree as pulse rate. Also described is a special technique for providing power to the heartbeat monitor that consists of a small diameter pin for providing an electrical connection between a battery and a circuit board comprising an IR transmitter and an IR receiver and one or more LED's. The battery unit is preferably on the inside of the earlobe and the circuit board is preferably on the outside of the earlobe. Also disclosed is a technique for charging the battery unit.

### BRIEF DESCRIIPTION OF THE DRAWINGS

FIGS. 1 and 2 are drawings from the '079 patent referred to above showing feature disclosed in that prior art patent.

FIG. 3 is a circuit diagram showing features of a working prototype of the present invention.

FIG. 3A is a version of FIG. 3 showing sections of the circuits shown enlarged in FIGS. 3A(1) through 3A(7)

FIGS. 3A(1) through 3A(7) each show a portion of the FIG. 3 circuit.

FIGS. 4A and 4B are prior art graphs that show heart rates of a female and male human engaged in love making.

FIGS. 5A and 5B show block diagrams of control circuits and logic for controlling a three-light embodiment of the present invention.

FIG. 6 is a drawing of an inexpensive IR transmitter and detector.

FIGS. 7A and 7B discloses a battery unit for providing power for the IR transmitter and the IR receiver and for one or more LED's.

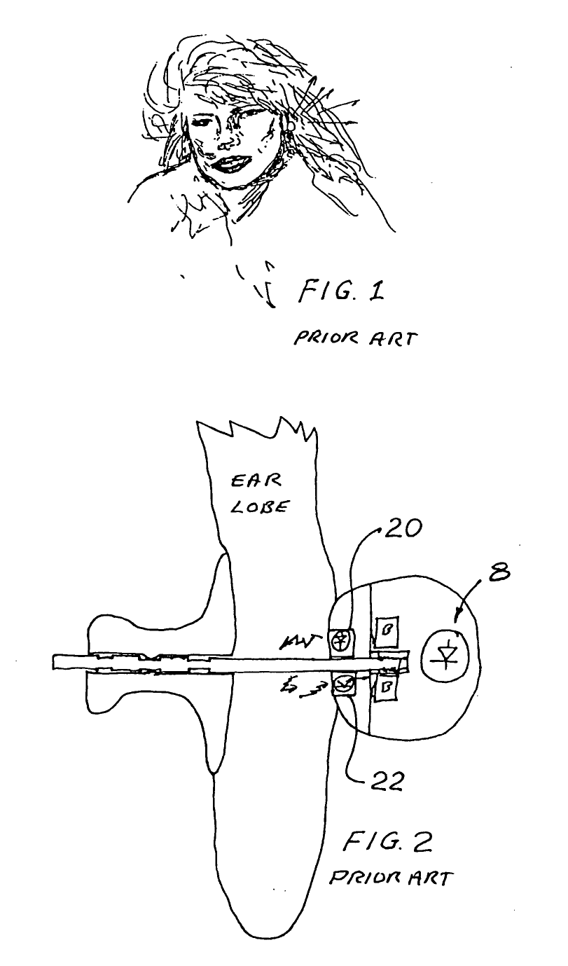

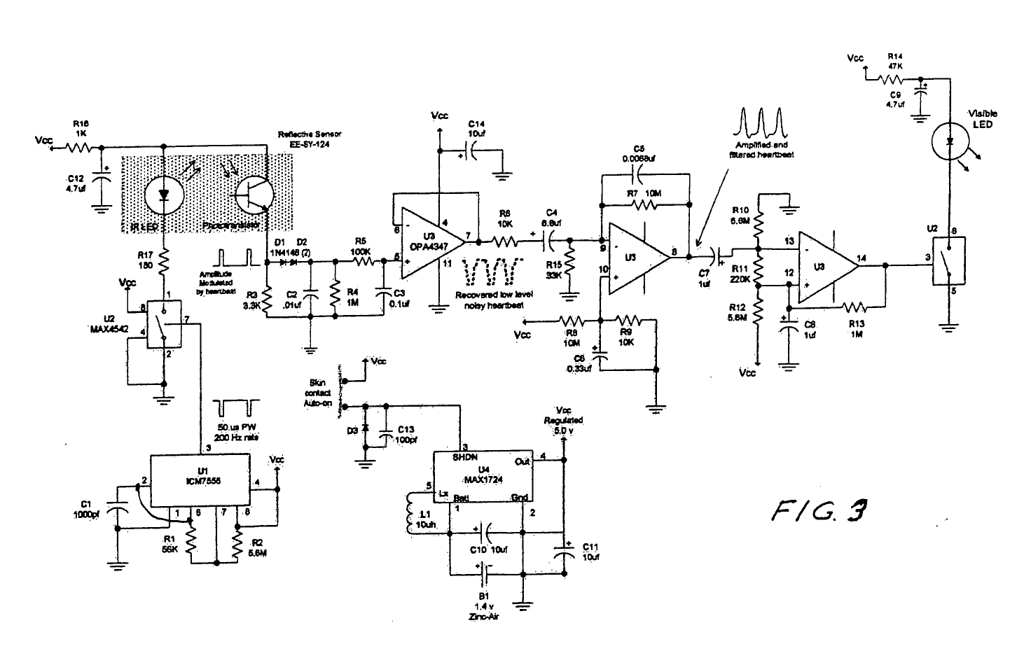

### DETAILED DESCRIPTION OF PREFERRED EMBODIMENTS

Prototype Embodiments

Applicants and their licensee have built and tested prototype-flashing earring that has demonstrated excellent performance of features of the present invention. A prototype earring was fabricated by modifying an earring having a magnetic clasp purchased from a Target department store and using inexpensive reflection IR emitter-sensors developed for card readers. Two magnets attract each other on both sides of a wearer's ear lobe. An IR transmitter, and IR receiver of the emitter-sensor and an on/off skin contact switch was mounted along with one of two magnets on one side of the ear lobe. The electronic parts for the earring are mounted on two small circuit boards contained in a heart shaped box hanging from the attaching magnetic clasps. The box has dimensions of about 1 inch×1 inch×¾ inch. The earring is fitted with a red Valentine shaped heart cover in which a visible light (red) emitting diode is mounted. Three hearing aid batteries also contained in the heart shaped box power this prototype earring. Only only a single battery powers other embodiments.

Circuit Diagram

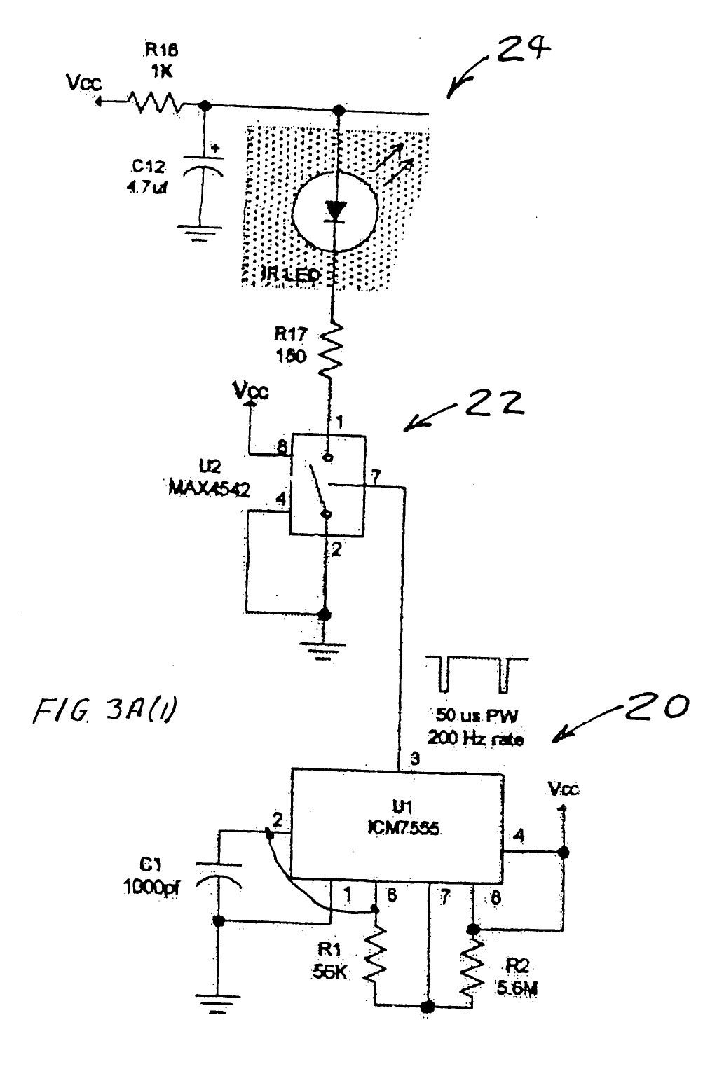

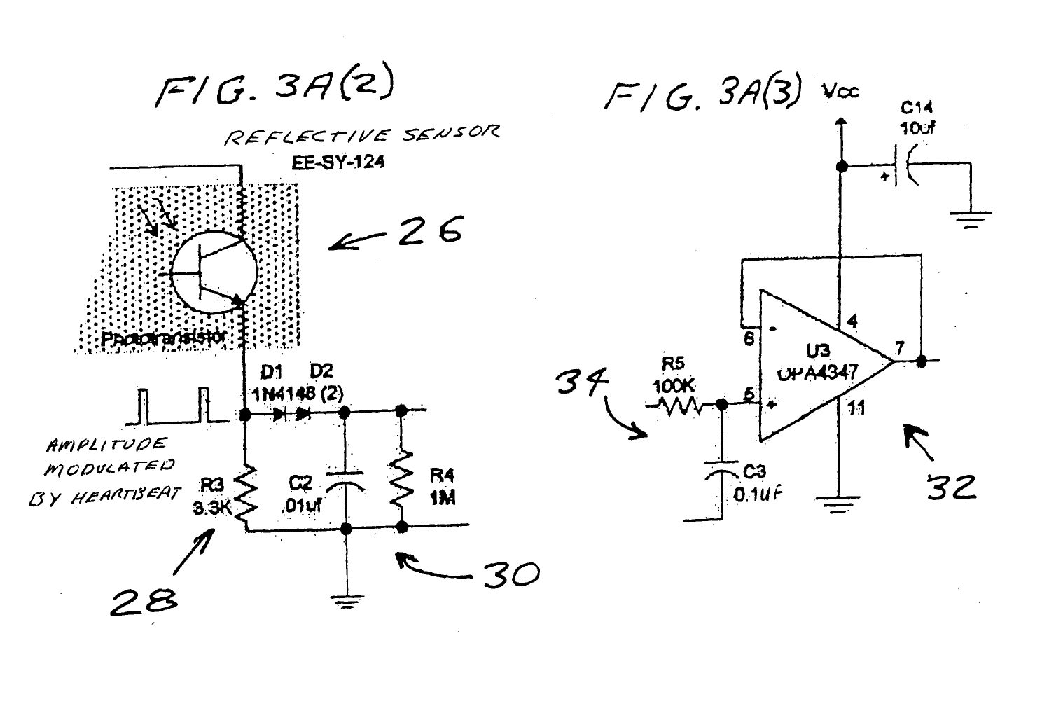

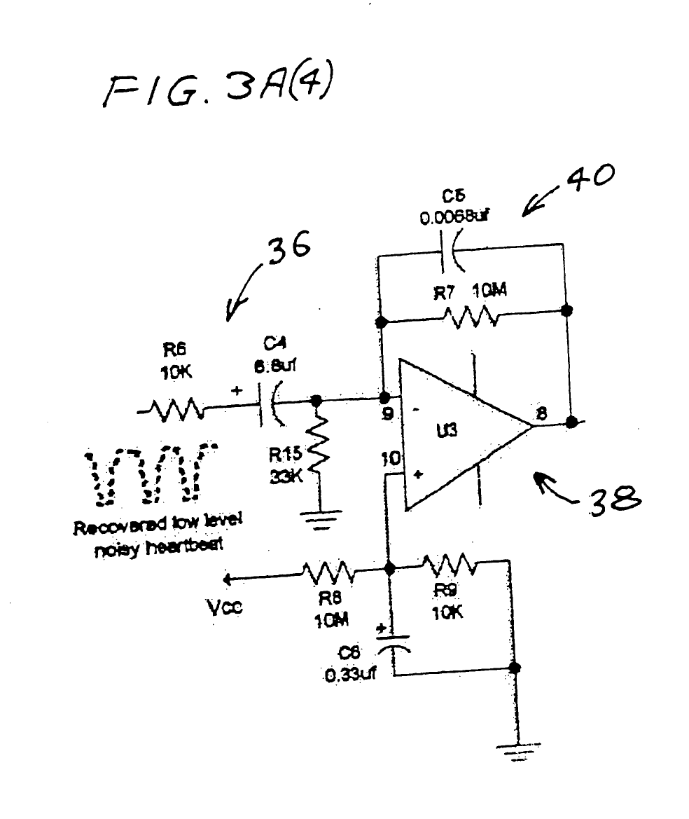

An electric circuit diagram of the prototype embodiment of the present invention actually built and tested by Applicants is shown in FIG. 3. This circuit diagram is also broken into seven parts as shown in FIG. 3A and FIGS. 3A(1) through 3A(7).

As shown in FIG. 3A(1) timer integrated circuit U1 with its associated components shown at 20 produces a pulse train of 500 microseconds pulse width pulses at a 200 Hz rate. These pulses activate the switch in IC U2 (a dual analog switch) 22 which permits current to flow in IR LED 24.

As shown in FIG. 3A(2), the emitted IR pulses enter the earlobe tissue and the light scattered back into the phototransistor 26 and is detected production a pulse amplitude modulated signal across R3 28. The network of D1, D2, C2 and R4 form a “track and hold” amplitude insulator 30.

As shown in FIG. 3A(3), integrated circuit U3 is a quad, OP Amp 32. The first amplifier is used as a voltage follower with high input impedance and a low output impedance which isolates the track and hold circuit from the following amplifier. R5 and C3 form a low pass filter 34 to reduce the high frequency noise from the pulse signal.

In FIG. 3A(4) R6 and C4 form a high pass filter 36 and OP amp 38 provides a gain of about 500 at the frequency range centered at about 2.5 Hz which is the frequency of interest. C5 along with the feedback resistor R7 forms another low pass filter 40. All the filters together form a narrow pass band filter centered at 2.5 Hz. The pass band is wide enough to detect heart rates between 40 pulses per minute and about 250 pulses per minute. The signal at pin 8 of OP amp is now the recovered heartbeat waveform.

In FIG. 3A(5) the recovered heartbeat waveform is AC coupled into the last OP Amp 42 which acts as a threshold detector. The network 44 of R10, R11 and R12 sets a threshold of about 0.2V. A signal above this amplitude causes the output (pin 14) 46 to go from 5 V to 0 V.

As shown in FIG. 3A(6) the second switch in U2 48 is closed upon detection of a beat, which causes current to flow in the visible LED 50. The decoupling network 52 comprising R14, C9 and R 16, C12 isolates the LED current transmits from the 08 Am PS.

In FIG. 3A(7) U4 is a DC-DC converter 54. A 1.4 V battery 56 is converted to a regulated 5V as shown at 60 to power the circuitry. It has a shutdown feature used to automatically turn on power upon skin contact as shown at 58.

Components of Prototype Are Off-the-Shelf

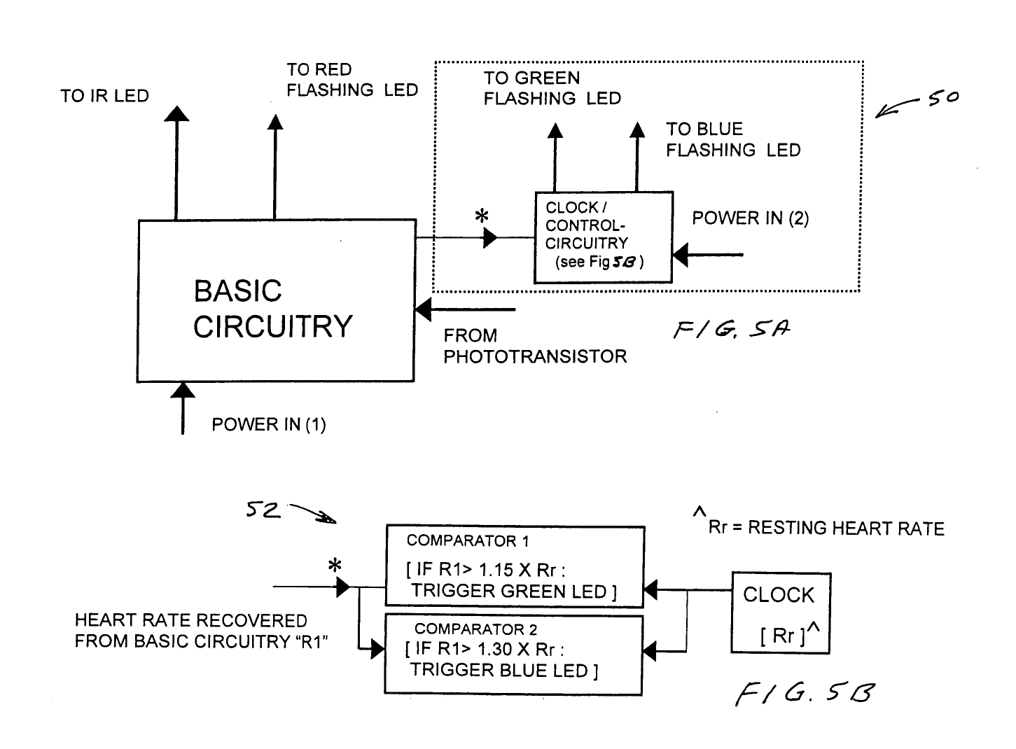

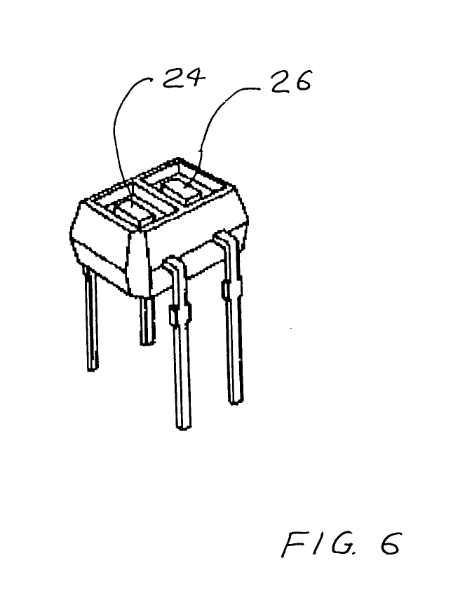

All of the electronic components of the above described prototype are off-the-shelf components available from vendors such as Radio Shack and Target with many locations, Allied Electronics with offices in Fort Worth Tex. and Digi-Key with offices in Thief River Falls, Minn. The IR transmitter and detector in the above embodiment is a very inexpensive device used for card reading an sells for $1.37 in quantities of 100. The unit operates at a wavelength of 940 nm which scatters well in and easily penetrates tissue as thick as the earlobe. Applicants have adopted it for their monitor with excellent results. A drawing of the IR transmitter and detector is shown in FIG. 6. Other parts are also very inexpensive so that Applicants expect to be able to produce their monitor for about $20 or less in large quantities. Therefore, Applicants expect enormous markets for the products of the present invention for medical, exercise, sports of all types and many other uses. As suggested above Applicants expect that their biggest market will be lovers.

Testing

The prototype earring has been thoroughly tested by Applicants and its excellent performance has been confirmed. One of the Applicants has tested the earring at a wide range of heart rates comparing the blinking of the LED with his own pulse with perfect match throughout the range.

Types of Jewelry Other than Earrings

Experimentation by Applicants have proven that the principals of the invention claimed in the '079 patent can be extended to other types of jewelry. Applicants have determined that heartbeat rings worn at other locations of the body perform just as well or better as compared to the earlobe. Other potential locations include the fingers, belly button, nose, toes, breast, and parts of the breast. Applicants have also determined that the flashing heartbeat monitor can be attached to skin as a patch with excellent performance. For example a heart shaped patch containing the electronic components described above can be applied on a skin region corresponding to the region of a person's heart.

Miniaturization

Although the prototype version of the invention actually built by Applicants is small enough to make a practical earring having substantial appeal, Applicants expect that much smaller earrings will have much greater appeal. Therefore, Applicants expect to have the electronics described converted to an application specific integrated circuit chip (an ASIC chip). These chips can be produced in volume production for less than $2 so that the cost of the heart monitoring jewelry could be as low a few dollars. Many ASIC manufacturers/designers are available which could convert the circuit shown in FIG. 3 into an ASIC chip mass producible for less than $2. These include Intrinsix Corp with offices in Westboro, Mass.; System to ASIC with offices in Bothell Wash. ASIC Northwest with offices in Portland, Oreg. and Arizona Microtek, Inc with offices in Mesa, Ariz. Another technique to miniaturize the jewelry of the present invention is by utilizing so called “surface mounting” techniques. These components are typically only about one-tenth to one fifth the size of conventional components. Thus, the prototype earring built and tested by applicants using off-the-shelf components, which is somewhat smaller than 1 inch cube, could be reduced in size to about one tenth or one fifth this volume using these surface mount techniques. Greater reductions are possible using the ASIC techniques but the surface mount approach generally requires a smaller front-end investment. Therefore, earrings of the present invention will be in sizes that are typical for earrings currently worn.

Three-Light Model

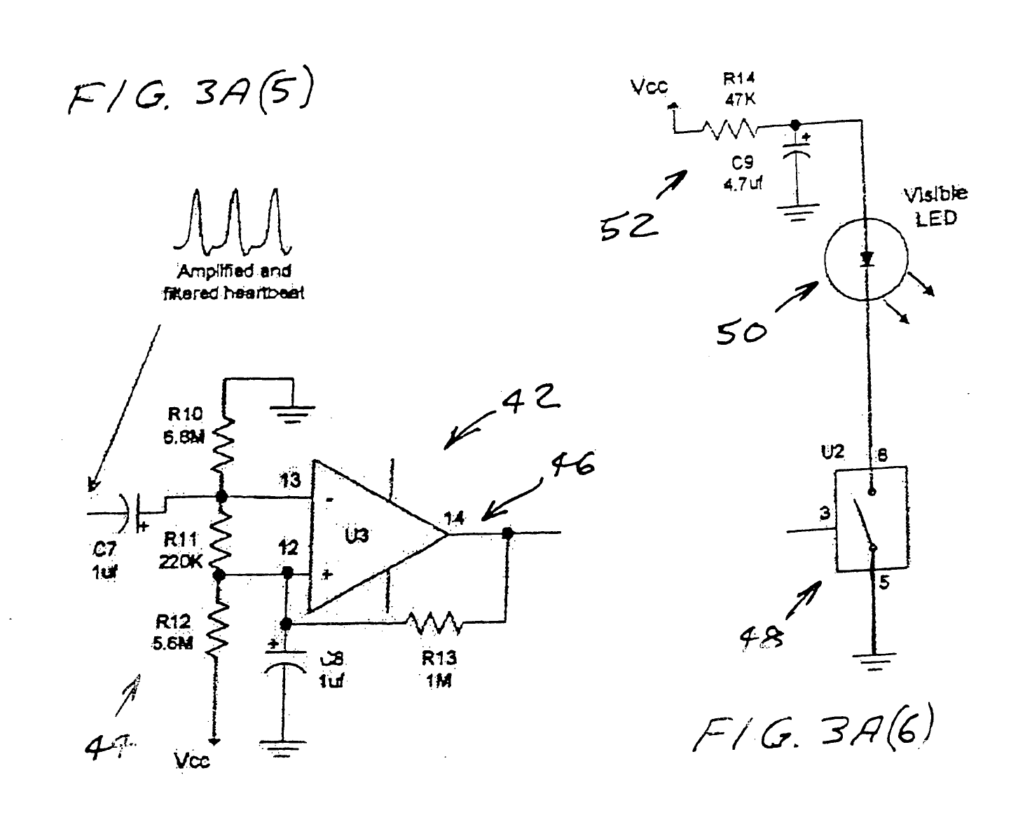

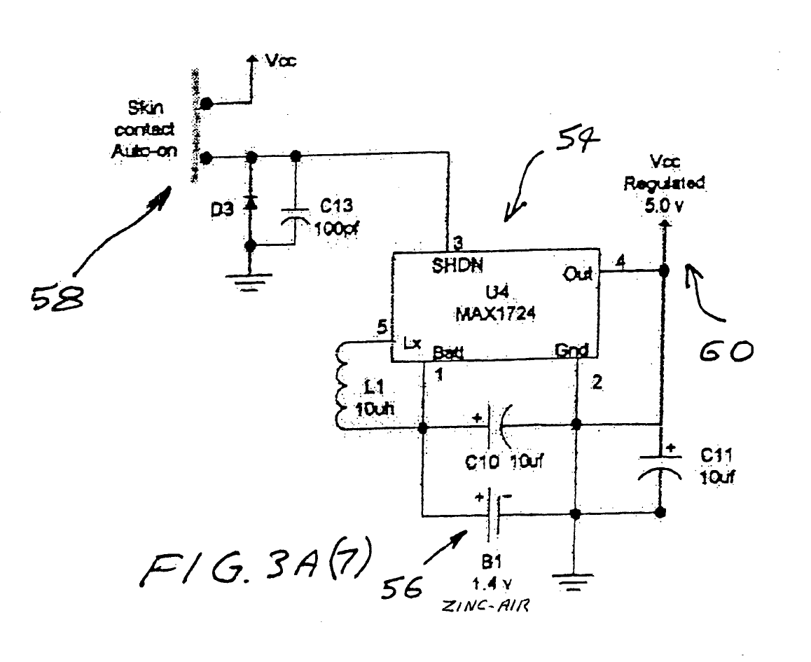

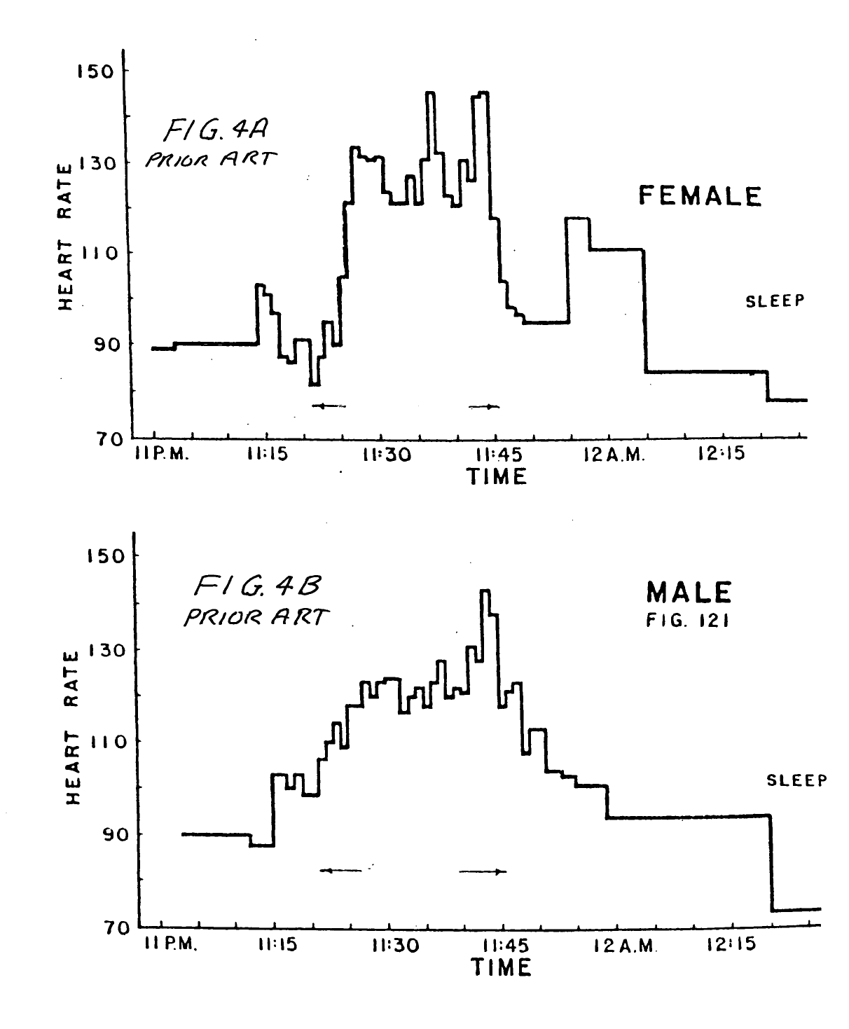

A preferred embodiment of the present includes an earring with three visible LED's, (red, green and blue) and a micro-processor which calculates pulse rate and causes the red LED to blink on each pulse, the green LED to blink on each pulse when the wearer's pulse rate is greater than a first threshold rate ( such as 115% of normal) and the blue LED to blink on each pulse when the wearer's pulse rate reaches a second threshold corresponding to extreme excitement (such as 130% of normal). These ranges would correspond to increased heart rates of typical persons engaged in exercise and love-making. In preferred embodiments the example percentages may be adjusted to span a wider range for persons such as younger persons and athletes who have a much greater range of heart rate. The monitor may also be self calibrating based on measurements of the wearer's heart rate over extended periods which would include periods of rest as well as periods of extreme exertion or excitement. FIG. 5A is a block diagram showing additional circuitry for the green and blue lights at 50, and FIG. 5B shows a simplified block diagram of the logic 52 for turning on the green and blue lights. In another preferred embodiment the earring is programmed to turn on the blue light when the heart rate increases by at least 15 percent within a period of 3 minutes or less. The reason for this approach will be obvious to the reader from a review of FIGS. 4A and 4B.

Blood Pressure Estimation and Indication

Blood pressure in humans varies in about the same degree as heart rate. Other preferred embodiments vary the brightness of the LED's (or other visible light) depending on the estimated blood pressure which also increases by about the same degree as pulse rate. In order to do this the detector monitors the intensity of measured light transmitted through tissue or reflected out of the tissue. The light transmitted or reflected is dependent on the amount of blood in the tissue which is in turn dependent on the blood pressure. In these preferred embodiment the LED will therefore not only blink with each heart beat but the intensity of the light will be roughly proportional to the blood pressure.

Battery Unit

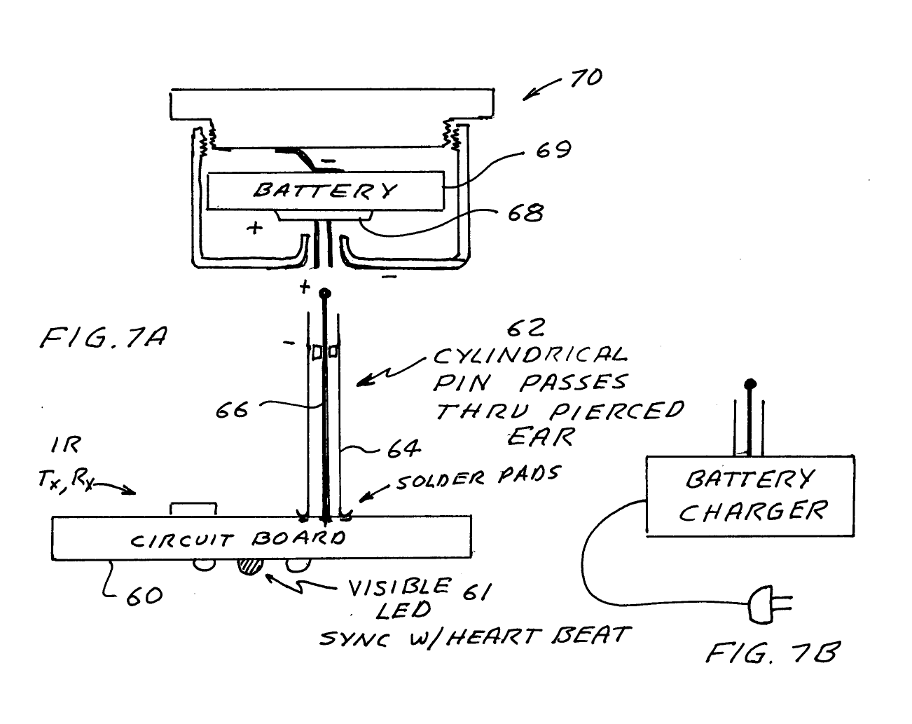

FIGS. 7A and 7B describe a technique for providing power to an earring heartbeat monitor. A circuit board 60 comprises an IR transmitter Tx and an IR receiver Rx and one or more LED light sources 61 as described above. The board 60 includes cylindrical pin 62 comprised of cylindrical conductor 64 having a diameter small enough to fit through the pierced ear of typical persons with pierced ears. Conductor 64 is treated as ground. The pin includes central conductor 66 that is connected to the positive terminal 68 of battery unit 70 as shown in FIG. 7A. Preferably, the board unit 60 is positioned on the outside of the earlobe and the battery unit 70 is positioned on the inside of the earlobe (i.e., between the user's earlobe and her neck). The reader should note that an advantage of this design is that power is drawn from the battery unit only when the earring is being worn. When the earring is not being worn (assuming that the battery is a rechargeable battery) the battery unit may be recharged as shown in FIG. 7B. The reader should note that the pin element could be a part of the battery unit instead of the board unit. The pin 62 could be held in place by a pressure fit or threads could be provided so that the battery unit 70 is threaded on to pin 62. Battery 69 could be a re-chargeable battery in which case a battery charge unit could be provided with a fitting similar to pin 62 for charging battery 69 with wall power as shown in FIG. 7B. Also, techniques could be added to accommodate various thicknesses of earlobes.

Although the present invention has been described in terms of particular embodiments, persons skilled in the art will recognize that many other embodiments using the principals of the invention are possible. Such variations include IR transmitters and receivers operating at different wavelengths which penetrate tissue and are absorbed in blood to a lesser or greater extent than the 940 nm light. Visible light components other than LED's could be used so long as they are small and efficient. The earring could include a tiny transmitter to transmit a signal to a nearby receiver. In a preferred embodiment the signal energizing the blue light could be transmitted to a nearby receiver that would then activate an audio receiver that would produce sounds such as church bells. Applicants have learned through testing of prototype earrings that bright sunlight adversely affects performance of the heartbeat earrings. Placing an infrared absorber between the earlobe and the clamp on the inside of the earlobe minimizes these adverse effects. Another solution to the sunlight problem is to design the earring so that the optical components of the earring are shielded from direct or reflected rays from the sun. Therefore, the scope of the invention should be determined by the appended claims and their legal equivalents.

1. Flashing jewelry comprising:
A) an infrared emitter positioned to emit infrared light into tissue of a wearer,
B) an infrared detector positioned to detect infrared light emanating from said tissue,
C) a power source for said emitter and said detector,
D) an electrical circuit for analyzing electrical signals from said detector to detect each beat of a wearers heart,
E) at least two visible light emitters,
F) a first trigger circuit for initiating electrical pulses to cause one of said visible light emitters to flash once for each heart beat,
G) a pulse rate calculation means for calculating the wearer's pulse rate, and
H) a second trigger circuit for initiating pulses to cause a second of said visible light emitters to flash once for each heart beat when said pulse rate exceeds a first predetermined rate.

2. Jewelry as in claim 1 and further comprising a third trigger circuit for initiating pulses to cause a third of said at least two visible light emitters to flash once for each heart beat when said pulse rate exceeds a second predetermined rate.

3. Jewelry as in claim 2 wherein said at least two visible light emitters are three visible light emitters emitting respectively red, green and blue light.

4. Jewelry as in claim 1 wherein said jewelry is an earring.

5. Jewelry as in claim 1 wherein said jewelry is a finger ring.

6. Jewelry as in claim 1 wherein said jewelry is attached to skin of said wearer by a patch.

7. Jewelry as in claim 1 wherein said jewelry is a patch in the shape of a heart.

8. Jewelry as in claim 1 wherein said at least one visible light emitter is three visible light emitters.

9. Jewelry as in claim 8 wherein said three visible light emitters are red green and blue emitters and said jewelry further comprises a means to determine heart rates of said wearer.

10. Jewelry as in claim 9 wherein said red emitter is programmed to flash with each heart beat, said green emitter is programmed to flash with each heart beat when the heart rate of the wearer is in excess of a first threshold in excess of the wearer's rest heart rate and said blue emitter is programmed to flash with each heart beat when the heart rate of said wearer is in excess of a second threshold in excess of said first threshold.

11. Jewelry as in claim 10 wherein said first threshold is at least 115% of the wearer's resting heart rate and said second threshold is at least 130% of wearer's resting heart rate.

12. Jewelry as in claim 1 wherein said electric circuit comprises and ASIC circuit.

13. Jewelry as in claim 1 wherein said electric circuit comprises surface mounted circuit.

14. Jewelry as in claim 3 and further comprising a transmitter for transmitting a signal to an audio device to initiate a sound when one of said thresholds are exceeded.

15. Jewelry as in claim 14 wherein said sound is church bells.

16. Jewelry as in claim 1 wherein said power source is a battery unit positioned on the inside of an earlobe and connected through an earlobe to a circuit board comprising said infrared emitter, said infrared detector and said at least two visible light sources
